(19) United States
(12) Patent Application Publication
(10) Pub. No.: US 2014/0066689 A1
(43) Pub. Date: Mar. 6, 2014

(54) Integrated Controller For Ventricular Assist Device

(71) Applicants: J. Bradford Rainier, Chelsea, MI (US); Alexander Medvedev, Ann Arbor, MI (US)

(72) Inventors: J. Bradford Rainier, Chelsea, MI (US); Alexander Medvedev, Ann Arbor, MI (US)

(73) Assignee: Terumo Kabushiki Kaisha, Tokyo (JP)

(21) Appl. No.: 13/602,788

(22) Filed: Sep. 4, 2012

(51) Int. Cl. A61M 1/12

(52) U.S. Cl. 600/16

(57) ABSTRACT

An integrated control module is provided for a ventricular assist device. A housing receives a power monitoring/switching circuit, a main receptacle, and a quasi-internal backup receptacle. The main receptacle within the housing receives a main battery. The main receptacle is configured for manual installation and removal of the main battery without requiring any separate tool. The quasi-internal backup receptacle within the housing receives a backup battery having a battery capacity less than the main battery. The backup receptacle includes a locking mechanism that requires use of a separate tool to unlock the locking mechanism for removing the backup battery. The backup battery is installed and removed without disturbing power supplied from a main battery in the main receptacle to the power monitoring/switching circuit.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to a left ventricular assist system (LVAS), and, more specifically, to an integrated controller that houses a main battery and a backup battery.

A heart pump system known as a left ventricular assist system can provide long term patient support with an implantable pump associated with an externally-worn control unit and batteries. The LVAS improves circulation throughout the body by assisting the left side of the heart in pumping blood. One such system is the DuraHeart® LVAS system made by Terumo Heart, Inc., of Ann Arbor, Mich.

A typical LVAS system employs a centrifugal pump implanted in the patient's chest, an inflow conduit coupling the pump inlet to the left ventricle, and an outflow conduit coupling the pump outlet to the aorta. During implantation, one end of the outflow conduit is mechanically fitted to the pump via a connector and the other end is surgically attached to the patient's ascending aorta by anastomosis. The pump is electronically controlled to provide a flow rate from two to eight liters per minute, for example. The controller typically contains a display screen for showing system status. The controller is connectable to a main console to allow a physician to set-up, adjust, and monitor the controller and pump units.

In addition to the controller, the extracorporeal portion of the circulatory support system includes batteries or other power supply. The batteries and controller are needed on a full time basis, so they are typically worn externally by the patient. A control and communication cable is connected between the implanted pump and the electronic controller via an exit site in the patient's skin. Once configured, the controller operates on a standalone basis and the entire system is portable to allow patient mobility.

The goal of the control unit is to autonomously control the pump performance to satisfy the physiologic needs of the patient while maintaining safe and reliable system operation. Since continuous operation is vital, conventional systems have been provided with at least two batteries so that there is backup capacity during mobile use. Typically one or two full-size batteries are installed in the system and carried by the patient. By using multiple main batteries, normal pump operation can continue beyond the charge time of one battery. Moreover, an alternate power source is thereby available during times that a discharged battery is being removed for recharging and replacement by a freshly charged battery. In addition, a lower capacity backup battery has also sometimes been used in conventional systems that is permanently mounted inside the control module and provides power when all other sources fail. The backup battery ensures sufficient power to operate alarms when main battery power may fail, and may also have capacity to run the pump for short periods.

For patient convenience and mobility, it is desirable to minimize the size of the external equipment. This goal can be met with an integrated controller that incorporates the main and backup batteries with all the control electronics. However, the battery combinations employed by conventional systems have not been successfully integrated. Furthermore, a problem involving the internal backup batteries has been that they eventually need to be replaced due to loss of charge-carrying capacity over time, and the control unit must be taken out of service in order to dismantle the controller module to access and replace the internal backup battery.

### SUMMARY OF THE INVENTION

In one aspect of the invention, an integrated control module is provided for a ventricular assist device. A housing receives a power monitoring/switching circuit, a main receptacle, and a quasi-internal backup receptacle. The main receptacle within the housing receives a main battery. The main receptacle is configured for manual installation and removal of the main battery without requiring any separate tool. The quasi-internal backup receptacle within the housing receives a backup battery having a battery capacity less than the main battery. The backup receptacle includes a locking mechanism that requires use of a separate tool to unlock the locking mechanism for removing the backup battery. The backup battery is installed and removed without disturbing power supplied from a main battery in the main receptacle to the power monitoring/switching circuit.

### BRIEF DESCRIPTION OF THE DRAWINGS

FIG. 1 is a diagram showing a ventricular assist device with a portion implanted in the body of a patient and a portion worn externally by the patient.

FIG. 2 is a perspective view of an integrated controller having installed main and backup batteries.

FIG. 3 is a perspective view of the integrated controller of FIG. 2 showing the installation/removal of the main battery.

FIG. 4 is bottom perspective view of the integrated controller of FIG. 2.

FIG. 5 is a bottom perspective view of the integrated controller of FIG. 4 showing the installation/removal of the backup battery.

FIG. 6 is a schematic, block diagram showing a power monitoring/switching circuit and a pump controller.

FIG. 7 is a perspective view of an alternate embodiment of an integrated controller.

### DETAILED DESCRIPTION OF PREFERRED EMBODIMENTS

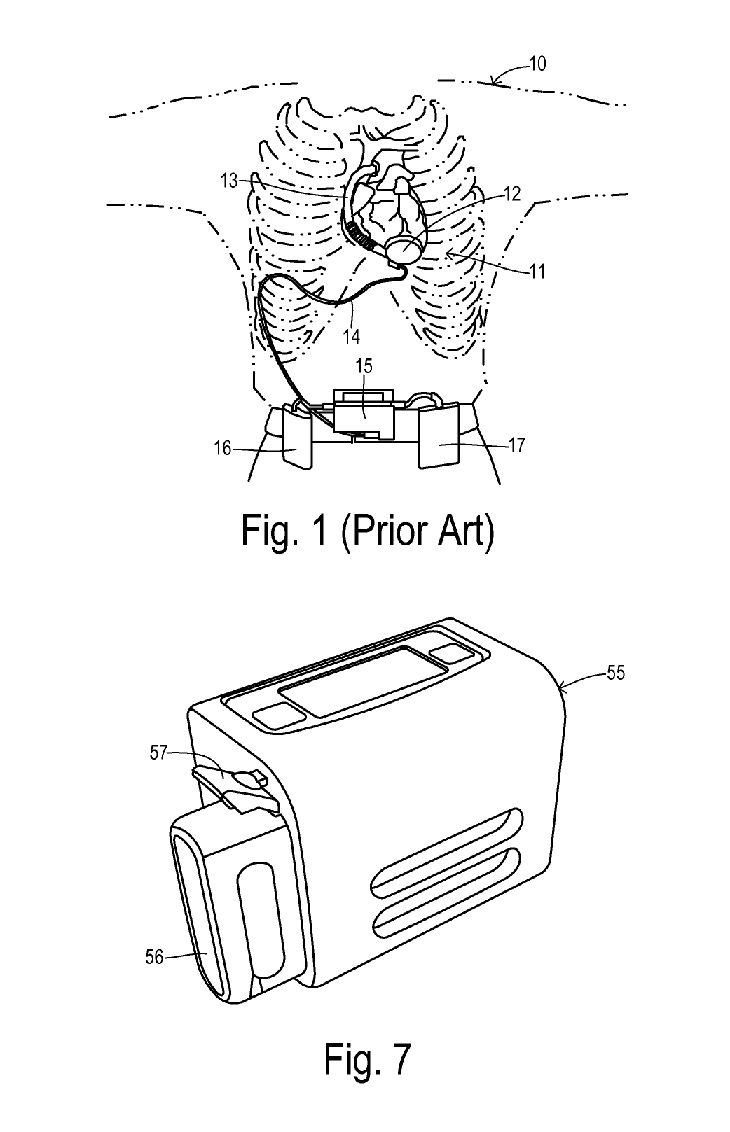

Referring to FIG. 1, a patient 10 is shown in fragmentary front elevational view. Surgically implanted into the patient's abdominal cavity or pericardium 11 is the pumping portion 12 of a ventricular assist device. An inflow conduit (not visible) penetrating the left ventricle conveys blood into the pumping portion 12, and an outflow conduit 13 conveys blood from the pumping portion 12 to the patient's ascending thoracic aorta. A power/communication cable 14 extends from pumping portion 12 outwardly of the patient's body via an incision to a compact controller 15. A power source, such as battery packs 16 and 17, worn on a belt about the patient's waist is connected with controller 15.

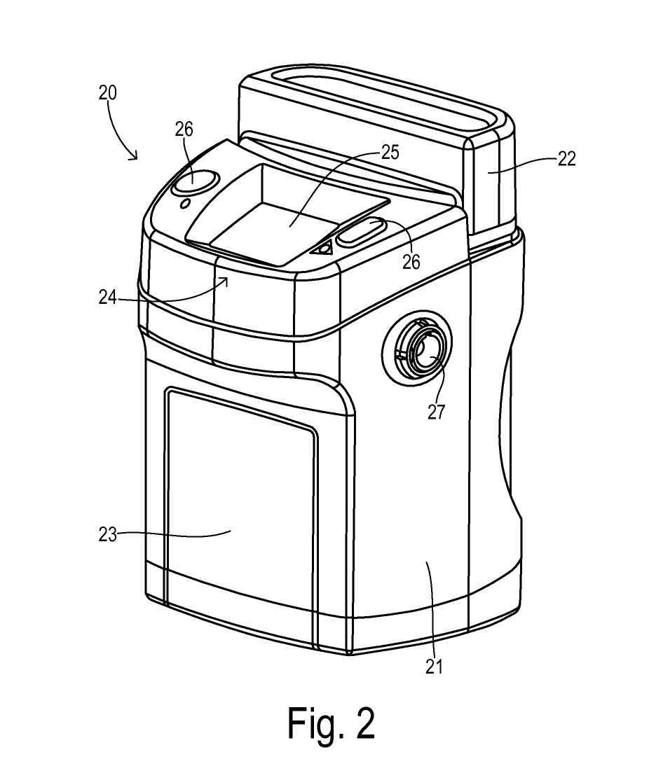

FIG. 2 shows an integrated controller and battery system 20 for performing control functions including controlling and monitoring operation of an implanted pump, managing power derived from either batteries or external AC/DC power supplies, and providing a user interface for the patient and/or caregiver. To ensure continual operation, a minimum of two sources of power need to be connected to the controller at any given time. System 20 is comprised of a single unit that integrates both the controller and battery sources in order to minimize the number of separate items that the patient must carry.

System 20 includes a housing 21 that incorporates an electronic controller (for performing typical controller functions including user interface, alarm, pump control and monitoring, power control and monitoring, etc.), a single main battery which is replaceable by the user, and a backup battery capable of supporting full operation for a short period of time (e.g., 30 minutes). Housing 21 receives a main battery 22 and a backup battery 23. A human machine interface (HMI) 24 includes a display 25 and control push buttons 26. A connector 27 interfaces the integrated control module to the power/communication cable that connects to the implanted pump.

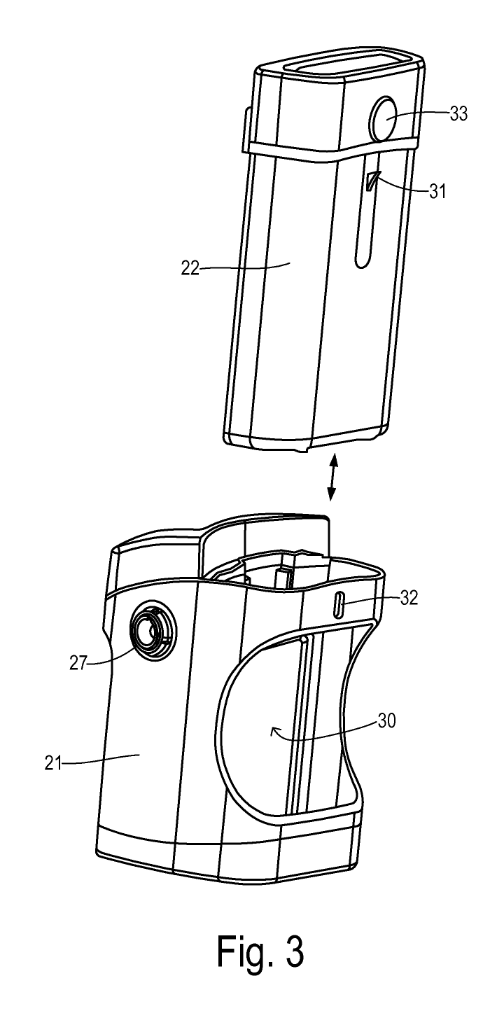

FIG. 3 shows a main receptacle 30 within housing 21 for receiving main battery 22. Main receptacle 30 is configured to accept a large size capacity battery for routine system operation, and is further configured to allow manual installation and removal of main battery 22 without requiring any separate tools. Thus, a user can easily swap out one main battery for another when necessary for recharging/replacement. For retention purposes, a manual interlock may be provided wherein a locking pin 31 on battery 22 is received in a slot 32 of receptacle 30. A retraction button 33 on battery 22 can be pressed in order to retract pin 31 to allow removal of main battery 22 from receptacle 30. Thus, the patient or a caregiver can easily remove main battery 22 for recharging and for replacement with a fully charged battery.

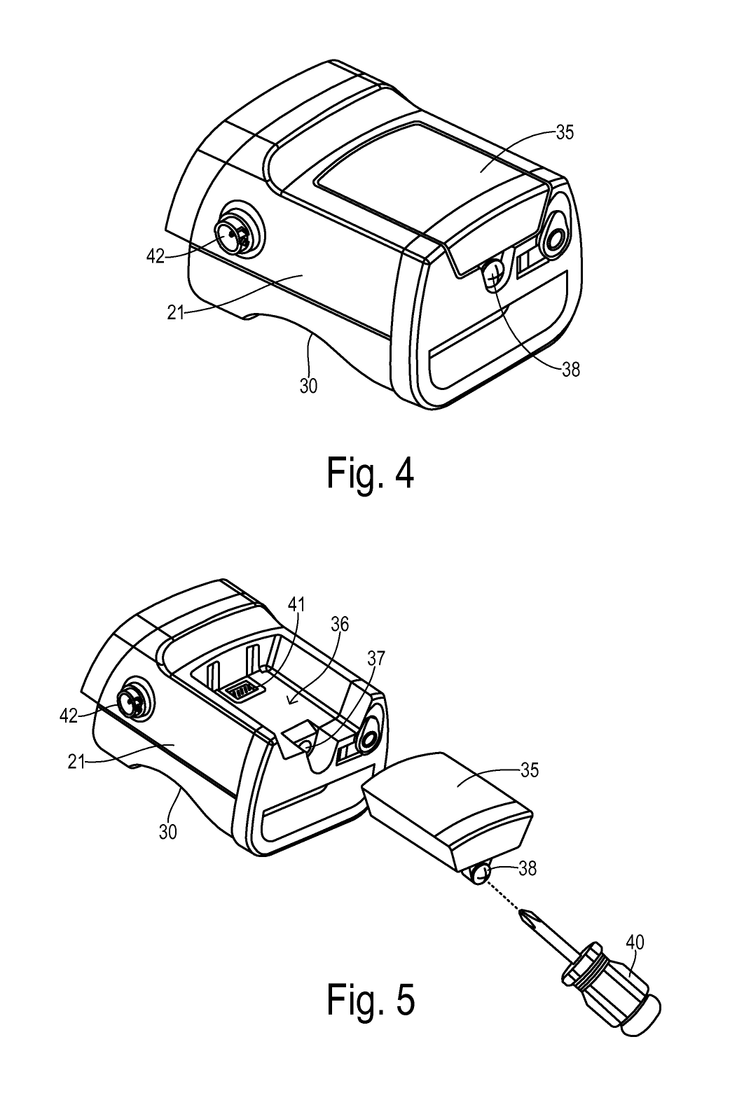

A benefit of the prior art internal backup battery which was housed in an inaccessible location within a control module was that the backup battery was always present and could not be inappropriately or mistakenly removed during times when the control module was in use. However, the control module had to be taken out of service whenever the backup battery needed replacement. To improve product longevity and flexibility, the present invention uses a quasi-internal backup battery as shown in FIGS. 4 and 5. Thus, a backup battery 35 is received in a quasi-internal backup receptacle 36 within housing 21. Backup receptacle 36 includes a locking mechanism comprised of a threaded socket 37 for receiving a threaded fastener 38 that is retained on backup battery 35. A separate tool such as a screw driver 40 is therefore required in order to unlock the locking mechanism when it is desired to remove the backup battery. A threaded connection is just one example of a possible locking mechanism. Any other mechanism such as a clip, lever, or other engaging mechanism can be employed provided that it cannot be unlocked except through use of a separate tool. The requirement for use of a separate tool ensures that the patient or caregiver must perform increased procedures in order to remove the backup battery as compared to removing the main battery. The increased difficulty reduces the possibilities of the backup battery being removed inappropriately or mistakenly. The term quasi-internal refers to the fact that even though the backup battery is accessible on the outside of the integrated controller, it is similar to an internal backup battery in that it cannot be removed without an appropriate tool.

Backup battery module 35 preferably contains an outer casing for forming a continuous surface with housing 21 when it is installed in receptacle 36. This adds to the impression that backup battery 35 should normally not be removed.

A power connector 41 within backup receptacle 36 meets with power terminals on backup battery 35 (not shown). An additional power connector 42 is provided for coupling the integrated controller to an external source of AC or DC power, such as when the patient is at home, traveling in an automobile, or in a hospital.

The locking mechanism shown in FIGS. 4 and 5 likewise requires use of the separate tool when installing backup battery 35 in receptacle 36. However, it is only necessary that the locking mechanism require separate tool use when unlocking to remove the backup battery. It would be permissible to permit battery installation without use of the tool since the objective of the quasi-internal backup battery is to increase the difficulty of placing the integrated controller into a condition in which a backup battery is not present.

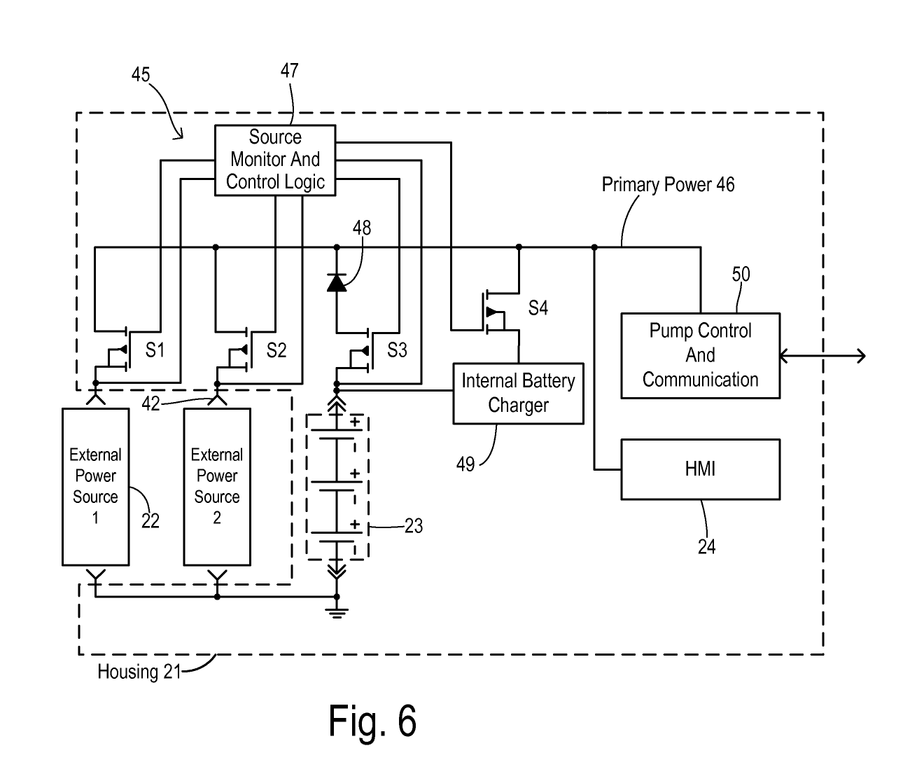

Another feature of the present invention is an ability to ensure that the backup battery can be removed and reinstalled without disturbing power supplied from a main battery in the main receptacle to a power monitoring/switching circuit installed within the housing. This allows the backup battery to be replaced without taking the controller module out of service. In FIG. 6, dashed box 21 represents the housing which contains a power monitoring/switching circuit 45, including a source monitor and control logic block 47, field effect transistors (FETs) S1, S2, and S3, and a diode 48 for selectably coupling power from backup battery 23 or external power sources #1 and #2 to a primary power bus 46. External power source #1 may be comprised of main battery 22, and external power source #2 may be comprised of an external AC/DC power supply connected to connector 42, for example.

Logic block 47 is respectively coupled to the supply terminals of external power sources #1 and #2 and to backup battery 23. Based on the detected state (i.e., presence and/or charge condition) of each potential power source and a predetermined priority for using the different power sources, control logic block 47 activates a corresponding one of FETs S1-S3 to couple the selected power source to power bus 46. A diode 48 couples the output of FET S3 to power bus 46 to prevent recharging of backup battery 23 directly from bus 46. Backup battery 23 is instead recharged using an internal battery charger circuit 49 receiving power from bus 46 via a FET S4. Logic block 47 controls the state of FET S4 such that backup battery 23 is recharged only when an external power source with sufficient capacity is present.

Power from primary power bus 46 is provided to HMI 24 and to a pump control and communication block 50 that interfaces via cable 14 to the implanted pump. In particular, power monitoring/switching circuit 45 is preferably configured to continuously supply power from an installed main battery during times that the backup battery is removed.

FIG. 7 shows an alternative embodiment in which an integrated control module 55 receives a main rechargeable battery 56 which is retained within a battery receptacle by a latch member 57. Latch member 57 engages when main battery 56 is fully inserted into the corresponding receptacle, and it helps ensure positive retention of battery 56 in the receptacle to reduce the possibility of an inadvertent disconnection. Member 57 can be manually shifted (i.e., without any separate tool) for releasing main battery 56 for replacement and recharging purposes.

What is claimed is:

1. An integrated control module for a ventricular assist device comprising:
a housing;
a power monitoring/switching circuit within the housing;
a main receptacle within the housing for receiving a main battery, wherein the main receptacle is configured for manual installation and removal of the main battery without requiring any separate tool; and
a quasi-internal backup receptacle within the housing for receiving a backup battery having a battery capacity less than the main battery, wherein the backup receptacle includes a locking mechanism that requires use of a separate tool to unlock the locking mechanism for removing the backup battery, and wherein the backup battery is installed and removed without disturbing power supplied from a main battery in the main receptacle to the power monitoring/switching circuit.

2. The integrated control module of claim 1 further comprising:
a pump controller within the housing connected to receive power from the power monitoring/switching circuit;
wherein the power monitoring/switching circuit is configured to continuously supply power from an installed main battery during times that the backup battery is removed.

3. The integrated control module of claim 1 wherein use of the separate tool is required to lock the locking mechanism for installing the backup battery.

4. The integrated control module of claim 1 wherein the locking mechanism is comprised of a threaded fastener that is movable between unlocked and locked positions by a screwdriver.

5. The integrated control module of claim 1 further comprising:
a backup battery module containing the backup battery and having an outer casing for forming a continuous surface with said housing when the backup battery module is installed in the backup receptacle.

6. The integrated control module of claim 1 further comprising:
an external power connector configured to receive external AC/DC power; and
an internal battery charging circuit coupled to the power monitoring/switching circuit for recharging the backup battery when external AC/DC power is present.
